(19) United States
(12) Patent Application Publication
(10) Pub. No.: US 2005/0164182 A1
(43) Pub. Date: Jul. 28, 2005

(54) Nucleotide analogues

(76) Inventors: Lea Pickering, Buckinghamshire (GB); Raj Odedra, Buckinghamshire (GB); Adrian Simmonds, Buckinghamshire (GB); Karin S Johnson, Hampshire (GB)

Correspondence Address: AMERSHAM BIOSCIENCES;PATENT DEPARTMENT, 800 CENTENNIAL AVENUE, PISCATAWAY, NJ, 08855, US

(21) Appl. No.: 10/496,761

(22) PCT Filed: Nov. 28, 2002
(86) PCT No.: PCT/GB02/05375

(30) Foreign Application Priority Data
Nov. 29, 2001 (GB) 0128526.1

(51) Int. Cl. C12Q 1/68; C07H 21/04; C07K 9/00

(52) U.S. Cl. 435/6; 536/24.3; 536/25.32; 530/322

(57) ABSTRACT

The invention relates to nucleosides comprising a reporter moiety which also functions to limit polymerase activity, characterised in that the reporter moiety is attached to the nucleoside through a linkage group cleavable by a hydrolase enzyme wherein the hydrolase enzyme is selected from the group consisting of esterases, phosphatases, peptidases, penicillin amidases, glycosidases and phosphorylases.

### FIELD OF INVENTION

The present invention relates to nucleoside and nucleotide analogues. In particular, the invention relates to nucleotide analogues comprising enzyme hydrolysable linkage groups attaching reporter moieties to the nucleotide.

### BACKGROUND OF THE INVENTION

Recent improvements in DNA sequencing techniques have sought to meet the increasing demands of large scale sequencing. Increasingly, methods in which the template nucleic acid molecules are attached to a solid surface are being developed (see, for example, U.S. Pat. No. 5,302,509 and U.S. Pat. No. 5,547,839). Such methods dispense with the need for an electrophoretic separation step and, with the use of optical detection technologies (see, for example, Nie et al. Annu. Rev. Biophys. Biomol. Struct. 1997, 26: 567-96), aim to allow sequencing information at the level of a single molecule to be obtained. This has the further potential for multiple samples to be analysed simultaneously.

One example of such methods is Base Addition Sequencing Scheme (BASS) (see, for example, Metzker et al., Nucleic Acids Res 1994, Vol. 22, No. 20; p. 4259-4267). BASS is a method involving the incorporation of nucleotide analogues which have been modified so as to comprise a blocking group which terminates DNA synthesis. A primer is annealed to a template bound to a solid support and sequence data obtained by repetitive cycles of incorporation of modified nucleotides. At each cycle, the incorporated base is identified in situ before being deprotected to remove the blocking group and allow the next cycle of DNA synthesis.

Methods such-as BASS rely on the use of nucleotide analogues that possess polymerase enzyme blocking (or terminator) groups at the 3′ hydroxyl position of the sugar on the nucleotide. Typically, the blocking group is a combined terminator and label/reporter moiety such that the incorporated nucleotide can be detected while the bulky label or reporter moiety itself fulfils the role of blocking a polymerase from any further DNA synthesis. Conveniently, as the terminator group is also the reporter moiety, a single reaction allows simultaneous removal of both functions thus allowing subsequent DNA synthesis and for incorporation of the next base to be read.

In order to allow subsequent rounds of DNA synthesis, these polymerase enzyme blocking groups are, typically, attached to the nucleotide via a lining group in such a way that they can be removed. However, conventional sequencing strategies require high temperatures of cycling (typically approximately 95° C. or above) which are associated with pH changes in the reaction mixture. Such conditions can cause reactivity of certain chemical bonds. Accordingly, the coupling methods for attaching blocking and labelling groups to nucleotides which have been used to date have focused on using those linking groups which can withstand changes in chemical conditions (such as temperature and pH). For example, the blocking and label groups can be attached via photosensitive linkage groups and thus cleavable by light irradiation (i.e. photochemical means, see, for example, WO 93/05183) or via chemical means.

WO 01/92284 describes nucleotides which comprise both a reporter moiety and a polymerase blocking group in which the reporter moiety does not also act as a polymerase enzyme blocking group. The polymerase enzyme blocking group is attached to the sugar by means of an enzyme cleavable linker.

Labeled reagents for nucleic acid sequencing are disclosed in EP 0252683. Igloi (BioTechniques, 1996, 21, 1084-1092) reports nucleotide-dye derivatives with utility in sequencing. In both cases, stable propynyl amide linking groups are used to attach the fluorescent dye to the base of these reagents.

Similarly, stable propynyl linking groups are used in dye-labelled nucleotides in WO 97/00967 and WO/88 10264.

The use of known nucleotide analogues suffers from a number of disadvantages.

There are many disclosures in the art of nucleosides having a reporter group attached via a linkage group to the sugar moiety (e.g. EP 0850949, WO 97/00967; WT 99/64437, U.S. Pat. No. 5,998,603).

Similarly, there are other reports in the art of nucleosides having a reporter group attached via a linkage group to the base (e.g. WO 88/10264, Conflone (J Heterocyclic Chem, 1990, 27, 3146), Sauer et al. (J Chem Soc, Faraday Trans, 1999, 1, 2471-2477), Prober et al (Science, 1987, 238, 336-341) and Tong et al (J Org Chem, 1993, 58, 2223-2231). However, in none of these documents is there any suggestion or evidence of cleavage of the linkage group by means of a hydrolase enzyme.

Known methods of removing the reporter/terminator groups require repeated insult by reactive chemicals or irradiation which can result in damage to the template DNA strand through reactions such as base transformation, crosslinking, or depurination.

Furthermore, by attaching the bulky reporter moiety in the 3′ position of the nucleotide, the ability of the DNA polymerase to recognise or tolerate the nucleotide may be reduced. In addition to being poorly incorporated, modified nucleotides may be inactive (i.e. not incorporated), inhibitory (i.e. inhibit DNA synthesis) or may result in an alteration of the polymerase enzyme fidelity.

Any one of, or a combination of, these effects will result in a reduced accuracy in the sequence data obtained and, in particular, a decreased signal-to-noise ratio will be found on detection. Moreover, this means that the amount of sequence data that can be obtained from successive rounds of enzyme incorporation and cleavage is limited. For example, if a combined error of approximately 3% in incorporation and cleavage were to accumulate, the result would be that sequence could only be obtained from 5 bases or fewer of the template DNA before the decreased signal to noise ratio made further sequencing impractical.

Accordingly, there is a need for improved nucleotide analogues. Such analogues may have one or more of the following attributes: tolerated by polymerases; stable during the polymerization phase; and the reporter groups can be removed efficiently under conditions which minimise damage to the template strand or template-primer complex. Preferably, the improved analogues display more than one of these features and most preferably they display all of these features.

It is thus an object of the invention to provide nucleoside and nucleotide analogues to which reporter moieties, which also limit polymerase-mediated incorporation, are attached via enzyme hydrolysable linkage groups. It is also an object of the invention to provide nucleoside and nucleotide analogues comprising reporter groups which are removable on enzyme hydrolysis of a labile moiety attached to the linkage groups of the analogues. Such nucleotide analogues are most suitable for using in sequencing reactions which involve an isothermic reaction and therefore do not involve exposure of the nucleotide analogues to high temperatures and to undesirable variations in chemical conditions. Under the conditions of suitable sequencing reactions, including array-based sequencing technologies (such as BASS), enzyme-cleavable groups will be essentially stable. The use of enzyme hydrolysable linking groups removes the need for harsh, template-damaging treatments to remove the reporter moieties.

### DESCRIPTION OF THE INVENTION

The present invention describes the use of linkage groups, cleavable by enzyme hydrolysis, to attach reporter moieties to nucleosides and nucleotides.

Accordingly, in a first aspect, the invention provides a nucleoside comprising a reporter moiety which also functions to limit polymerase activity, characterised in that the reporter moiety is attached to the base of the nucleoside through a linkage group cleavable by a hydrolase enzyme, wherein the hydrolase enzyme is selected from the group consisting of esterases, phosphatases, peptidases, penicillin amidases, glycosidases and phosphorylases.

In contrast to enzyme cleavable nucleosides of the prior art, the reporter moiety serves the dual function of providing a detectable label and of limiting polymerase enzyme activity. Thus nucleosides of the present invention do not comprise a separate reporter moiety and a separate polymerase enzyme blocking group.

A hydrolase is defined as any member of the class of enzymes that catalyse the cleavage of a chemical bond with the addition of water.

In a second aspect, the invention provides a compound of Formula I:
R-L1-L2-L3-L4-L5-BASE-SUGAR (I)
wherein
R is a reporter moiety
L1 and L5 are optional linkage groups each containing one or more atoms comprising hydrocarbon chains which may also contain other atoms such as N, O and S.
L2 and 14 are optional linkage groups comprising 1 or more amino acid residues.

L3 is a linkage group that is susceptible to enzymic hydrolysis by a hydrolase enzyme, wherein hydrolytic cleavage may be within the group or adjacent to the group and characterised in that the hydrolase enzyme is selected from the group consisting of esterases, phosphatases, peptidases, penicillin amidases, glycosidases and phosphorylases.

Suitably, enzymic hydrolysis of the linkage group L3 produces an unstable moiety which undergoes chemical hydrolysis.

Suitable bases comprise purines or pyrimidines and, in particular, any of the bases A, C, G, U and T or analogues thereof.

Suitably, the sugars comprise ribose or deoxyribose or analogues thereof. Thus ribonucleotides and deoxyribonucleotides are envisaged together with other nucleoside analogues.

Suitably, a mono-, di- or tri-phosphate group is attached to the sugar. In a particularly preferred embodiment, a triphosphate group is attached to the sugar.

In a preferred embodiment, the composite linker group L1 to L5 may be a chain of 10 to 200 bond lengths and may include atoms selected from carbon, nitrogen, oxygen and sulphur atoms, the linker group may be rigid or flexible, unsaturated or saturated as is well known in the field. The composite linker group may further incorporate one or more amino acids joined by peptide bonds. The incorporation of amino acids can be through the incorporation of amino acid monomers or oligomers using standard amino acid chemistry (see, for example, “Synthetic Peptides—A Users Guide” Ed. G. A. Grant; 1992).

Hydrolysis of L3 by a hydrolytic enzyme selected from the group consisting of esterases, phosphatases, peptidases, glycosidases, penicillin amidases and phosphorylases results in the polymerase enzyme reporter moiety (R) becoming detached from the compound.

In a preferred embodiment, the linkage group 13 is cleavable by hydrolase enzymes selected from the group consisting of esterases, phosphatases, peptidases, glycosidases and phosphorylases. Preferred peptidases include subtilisin, proteinase K, elastase, neprilysin, thermolysin, papain, plasmin, trypsin, enterokinase and urokinase. Suitable enzymes are those that are reactive under mild conditions (see Handbook of Proteolytic Enzymes, Barrett et al., ISBN 0-12-079370-9). In a particularly preferred embodiment, the enzyme-hydrolysable group is cleavable by penicillin amidase.

Preferably, L3 is a peptide selected from the group consisting of alanine-alanine-alanine, alanine-alanine-leucine, glycine-leucine-serine, glycine-serine-alanine-alanine-leucine and glycine-alanine-glycine-leucine.

Esterases catalyse the general reaction set out below in Reaction Scheme 1:

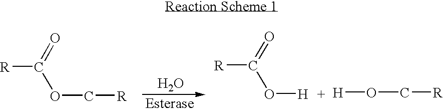

Thus, in a further preferred embodiment of the second aspect, L3 comprises an ester group.

Non-specific esterase activity is associated with a number of enzyme systems. This activity has been associated with both physiological function and drug metabolism. Such a non-specific carboxylesterase activity can be used to modify molecules in vitro. However, the lack of stability of carboxyesters at moderately high pH and elevated temperatures can make them unsuitable for generating stable reagents for nucleic acid applications.

In a preferred embodiment highly stable peptide bonds are utilised as specifically cleavable groups. The linkage groups may be digested with a suitable peptidase as shown in Reaction Scheme 2 to remove the reporter moiety without damaging the template strand or the template/primer complex. Following deprotection, further DNA synthesis can take place leading to the next cycle of labelled analogue addition.

Where R′ and R″ both represent one or more amino acid residues, then peptidases catalyse the following general reaction set out in Reaction Scheme 2:

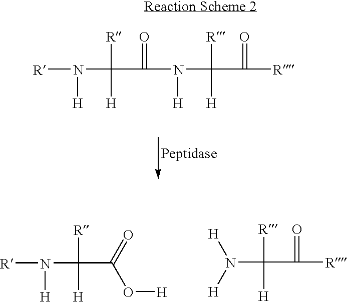

Suitably, enzymic hydrolysis of the linkage group L3 produces an unstable moiety which undergoes chemical hydrolysis.

A linker may be assembled such that L3 comprises a moiety that is not part of the linker backbone and is not directly bonded to the base or the reporter groups (through, for example, linkage groups L2 or L4), which may be enzymatically hydrolysed to a chemically unstable form. Thus, on removal of such a moiety by a hydrolytic enzyme, the unstable linkage group formed rapidly undergoes chemical hydrolysis to facilitate cleavage of the linker. This is distinct from direct enzymatic cleavage of the linker by the enzyme in that the covalent bonds broken by the enzyme do not form part of the contiguous chain of covalent bonds comprising the linker between the nucleoside and the label. An example of this type of “remote cleavage” would be when L3 consists of an N-phenylacetyl amino acetal. The enzyme penicillin amidase specifically recognises the phenylacetyl portion of the molecule and hydrolyses the amide bond, giving phenyl acetic acid and a hemiaminal (see, for example, WO 97/20855). The hemiaminal is highly susceptible to acid or base catalysed hydrolysis in the absence of the enzyme. Thus if L3 comprises such a unit, treatment with penicillin amidase would render the linker chemically unstable, resulting in cleavage on base or acid catalysed hydrolysis. This is depicted in Reaction Scheme 3:

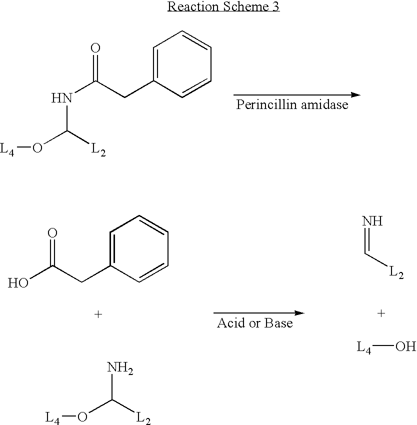

Other systems that work on this principle could be devised and incorporated into the design of any linker. The particular advantage of this type of linker cleavage is that the enzyme recognition and cleavage site is remote from the linker cleavage site and may be less affected by the proximity of the other components of the linker, the label or the nucleoside.

Accordingly, in a particularly preferred embodiment, L3 comprises a penicillin amidase cleavage site. Penicillin amidase is also known as penicillin aminohydrolase (EC 3.5.1.11).

Suitable methods for attaching a linker comprising an enzyme cleavable group to a base moiety are described, for example, in Cavallaro et al. Bioconjugate Chem. 2001, 12, 143-151. Further methods are described in Langer et al., Proc Natl Acad Sci USA; 1981, 78, 6633-6637; Livak et al., Nucleic Acids Res, 1992, 20, 4831-4837 and Gebeyehu et al., Nucleic Acids Res, 1987, 15, 4513-4534.

A suitable reporter moiety, R, may be any one of various known reporting systems. It may be a radioisotope by means of which the nucleoside analogue is rendered easily detectable, for example 32P, 33P, 35S incorporated in a phosphate or thiophosphate or H phosphonate group or alternatively 3H or 14C or an iodine isotope. It may be an isotope detectable by mass spectrometry or NMR. It may be a signal moiety e.g. an enzyme, hapten, fluorophore, chromophore, chemiluminescent group, Raman label, leucodye, electrochemical label, or signal compound adapted for detection by mass spectrometry.

In a preferred embodiment, the reporter moiety has fluorescent properties and can be detected using a sensitive fluorescence detector. It may be a fluorophore, for example, selected from fluoresceins, rhodamines, coumarins, BODIPY® dyes, phenoxazine dyes, cyanine dyes and squarate dyes (described, for example, in WO 97/40104). Preferably, the dyes are acridone derivatives, as described in WO 02/099424 and WO 02/099432. Most preferably, the reporter moiety is a cyanine dye. The Cyanine dyes (sometimes referred to as “Cy dyes™”), described, for example, in U.S. Pat. No. 5,268,486, are a series of biologically compatible fluorophores which are characterised by high fluorescence emission, environmental stability and a range of emission wavelengths extending into the near infra-red which can be selected by varying the internal molecular skeleton of the fluorophore.

In a preferred embodiment, the modified nucleotide remains compatible with elongation enzymology, i.e. it can still be incorporated by a polymerase. Procedures for selecting suitable nucleotide and polymerase combinations will be readily adapted from Metzker et al., Nucleic Acids Res 1994, Vol. 22, No. 20, 4259-4267. In particular, it is desired that a selected polymerase be capable of selectively incorporating a nucleotide.

In another preferred embodiment, the reporter group, R, restricts further elongation of the polymer to a limited number of additions by a polymerase once the nucleotide of the present invention has been incorporated by a selected polymerase in specified polymerase enzyme conditions.

The invention further provides a chemical intermediate selected from the group consisting of: 5-N-(N-Trifluoroacetyl-β-alanyl)propargylamino-2′-deoxyuridine; 5-N-(β-alanyl)propargylamino-2′-deoxyuridine; 5-N-(N-Fluorenylmethyloxycarbonyl-Gly-Gly-Leu-β-alanyl)propargylamino-2′-deoxyuridine; 5-N-(-Gly-Gly-Leu-β-alanyl)propargylamino-2′-deoxyuridine and 5-N-[N-(6-Fluorescein-5(and-6)carboxamidohexanoyl)-Gly-Gly-Leu-β-alanyl]-propargylamino-2′-deoxyuridine.

In a third aspect, the invention provides a chemical intermediate of Formula II
R-L1-L2-L3-L4-L5 (II)
wherein
R is a reporter moiety
L1 and L5 are optional linkage groups each containing one or more atoms comprising hydrocarbon chains which may also contain other atoms such as N, O and S,
L2 and L4 are optional linkage groups comprising 1 or more amino acid residues, and

L3 is a linkage group that is susceptible to enzymic hydrolysis by a peptidase enzyme. Chemical intermediates of Formula II are of use as dye-linker groups in the synthesis of compounds of Formula I.

In a preferred embodiment, L3 is selected from the group consisting of alanine-alanine-alanine, alanine-alanine-leucine, glycine-leucine-serine, glycine-serine-alanine-alanine-leucine and glycine-alanine-glycine-leucine.

Preferably, the reporter R (or dye) is selected from the group consisting of of fluoresceins, rhodamines, coumarins, BODIPY® dyes, phenoxazine dyes, cyanine dyes, acridone dyes and squarate dyes.

In a fourth aspect, the invention provides a compound comprising 5-N-[N-(6-Fluorescein-5(and-6) carboxamidohexanoyl)-Gly-Gly-Leu-β-alanyl]-propargylamino-2′-deoxyuridine triphosphate.

In a fifth aspect, the invention provides a set of nucleotides characterised in that the set contains at least one compound of Formula 1 having a mono-, di- or triphosphate group attached to the sugar. Preferably, the set comprises each of the four natural bases A, G, C and T and analogues thereof.

In a preferred embodiment of the fifth aspect the set of nucleotides further comprises at least two compounds as described above having different bases, characterised in that each compound has a different reporter moiety, R. Thus, for example, the set of nucleotides may comprise compounds with bases A and G, wherein the compound with base, A, has a first reporter moiety (R¹) and the compound with base, G, has a second reporter moiety (R²), and the first and second reporter molecules are distinguishable from each other.

In another preferred embodiment of the fifth aspect, the set of nucleotides comprises four compounds as described above characterised in that each compound has a different base such that each of the bases A, G, C and T, or analogues thereof, are present and each of the four compounds has a reporter moiety which is distinguishable from the reporter moiety of each of the compounds having the other three bases.

In a sixth aspect, the invention provides a method for nucleic acid molecule sequencing comprising the steps of:
a) immobilising a complex of a primer and a template to a solid phase
b) incubating with a polymerase in the presence of a compound of Formula I having a mono-, di- or triphosphate group attached to the sugar.

In one embodiment of the sixth aspect, the complex of primer and template can be preformed by incubation under appropriate hybridisation conditions before immobilising the complex onto a solid phase. In another embodiment, the primer or the template can be immobilised onto a solid phase prior to formation of the complex by introduction of the appropriate hybridisation partner (i.e. template or primer, respectively). In yet another embodiment, the complex immobilised onto the solid phase can be a single nucleic acid molecule comprising both “primer” and “template”; for example, the immobilised poly- or oligo-nucleotide can be a hairpin structure.

Suitable polymerases are enzymes that perform template-dependent base addition including DNA polymerases, reverse transcriptases and RNA polymerases. Suitable native or engineered polymerases include but are not limited to T7 polymerase, the Klenow fragment of E. coli DNA polymerase I which lacks 3′-5′exonuclease activity, E. coli DNA polymerase III, Sequenase™, φ29 DNA polymerase, exonuclease-free Pfu, exonuclease-free Vent™ polymerase, Thermosequenase, Thermosequenase II, Tth DNA polymerase, Tts DNA polymerase, MuLv Reverse transcriptase or HIV reverse transcriptase. The selection of an appropriate polymerase depends on the interaction between a polymerase and the specific modified nucleotide (as described by Metzker et al., Nucleic Acids Res 1994, Vol. 22, No. 20; p. 4259-4267).

Nucleotides comprising hydrolase-cleavable linkage groups such as caxboxyl ester attachment groups are suitable for use in sequencing reactions used in array based sequencing, such as BASS. Such reactions are isothermic, unlike cycle sequencing, so allowing much better control of reaction conditions. In particular, the sequencing reaction takes place at relatively low temperatures (typically less than 70° C.) thus enabling enzyme-cleavable linkage groups, such as the carboxyl ester attachment, to remain stable under these sequencing reaction conditions. Accordingly, polymerases which may be useful in the sixth aspect of the invention include thermostable polymerases and non-thermostable polymerases.

In a preferred embodiment of the sixth aspect, the method further comprises the steps of:
c) detecting the incorporation of a compound of Formula I having a mono-, di- or triphosphate group attached to the sugar
d) incubating in the presence of enzyme under suitable conditions for enzymatic cleavage of the enzyme-cleavable group L3

Preferably, the hydrolase enzyme is selected from the group consisting of esterases, phosphatases, peptidases, penicillin amidases, glycosidases and phosphorylases.

Suitable conditions for enzyme hydrolysis of the enzyme-cleavable groups will depend on the nature of the enzymes involved. Enzymes such as carboxyesterases are active under a broad range of conditions and do not require co-factors. Commercially available carboxyesterases will hydrolyse esters under mild pH conditions of between pH 7.0 and pH 8.0. e.g. 0.1M NaCl, 0.05M Tris.HCl, pH 7.5.

Suitable peptidases may be selected from the group consisting of subtilisin, proteinase K, elastase, neprilysin, thermolysin, papain, plasmin, trypsin, enterokinase and urokinase. Suitable conditions for cleavage by peptidases are exemplified in Example 5 below.

In another embodiment of the sixth aspect, the method further comprises:
e) repeating steps a)-d)

In a preferred embodiment of the sixth aspect, the enzyme in step d) is penicillin amidase.

In a preferred embodiment of the sixth aspect, the incorporation of the compound is determined by the detection of a single reporter group attached to the compound.

Briefly, sequencing reactions using modified nucleotides in accordance with the second aspect of the invention may be performed as follows. Primer template complexes are immobilised to a solid surface and contacted with modified nucleotides in the presence of a suitable buffer also containing a polymerase, such as Klenow fragment of E. coli DNA polymerase I which lacks 3′-5′exonuclease activity, and a commercially available pyrophosphatase. The reaction is incubated under suitable conditions for a polymerase-mediated base addition reaction followed by the removal of non-incorporated nucleotides and enzymes by washing with a wash buffer. Suitably, the wash buffer contains a buffering agent, such as an organic salt, to maintain a stable pH of approximately pH 6 to pH 9 and possibly also comprises monovalent or divalent cations and a detergent so as to eliminate non-covalently bound molecules from the solid surface. Where the modified nucleotides comprise a fluorescent reporter molecule, incorporated nucleotides are detected by measuring fluorescence. Following identification, the templates are contacted with a buffered solution containing an excess of a protein displaying the appropriate enzyme activity and incubated under conditions for enzyme cleavage activity. For example, where the enzyme-cleavable group linking the reporter moiety to the nucleotides is a peptide group, the solution contains an excess of a protein displaying peptidase activity. Following enzyme activity, the soluble products of enzymatic cleavage are eliminated by washing as above. Following the washing step, the immobilised template is washed with an excess of buffer used for the polymerase reaction and the steps of polymerase-mediated base addition, detection of incorporated nucleotide and enzyme-cleavage activity are repeated to obtain further sequence data.

### SPECIFIC DESCRIPTION

For the purposes of clarity, certain embodiments of the present invention will now be described with reference to the following figures and examples:

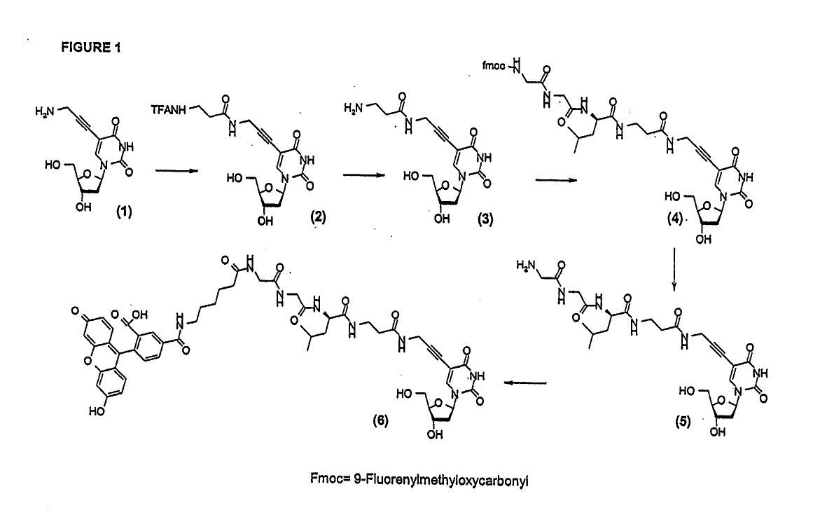

FIG. 1 (Example 1) shows a reaction scheme for synthesising a compound of Formula I.

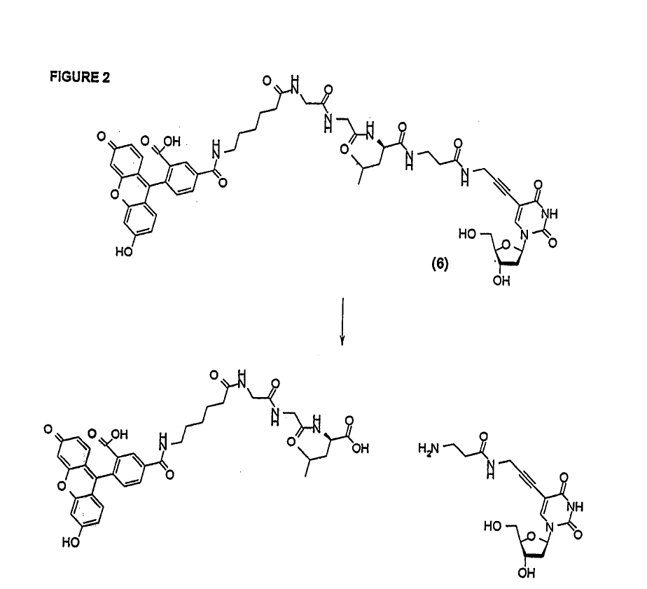

FIG. 2 (Example 2) depicts enzyme cleavage of a compound of formula I.

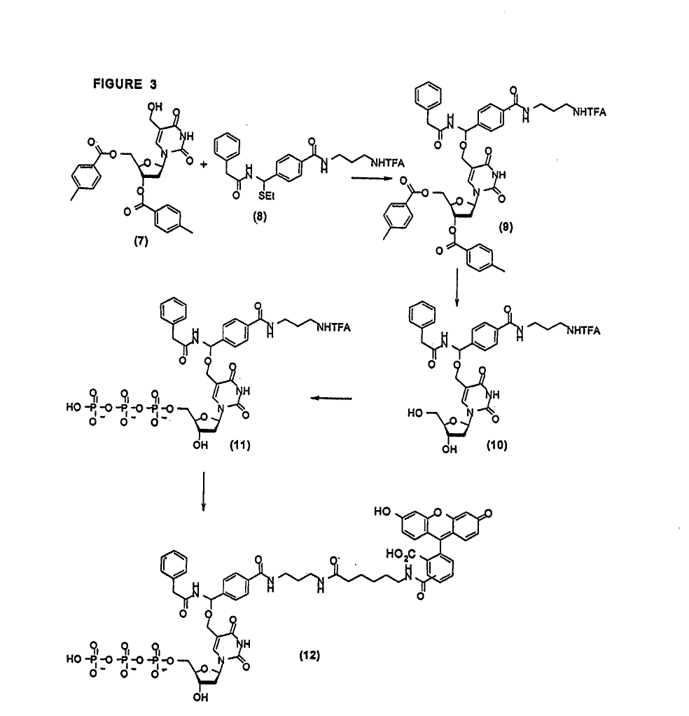

FIG. 3 (Example 3) shows a reaction scheme for the synthesis of a nucleotide with a penicillin amidase cleavable linker.

Example 4 describes the incorporation of a compound of Formula I by a DNA polymerase.

Example 5 describes protease mediated cleavage of FamHex-GGL-β-A-2′dU

Example 6 describes the preparation of dye-labelled nucleosides with protease cleavable linkers.

### EXAMPLE 1

A reaction scheme for the synthesis of an example of a compound of Formula I containing a peptide-based linker is set out in FIG. 1.

### i) 5-N-(N-Trifluoroacetyl-β-alanyl)propargylamino-2′-deoxyuridine (2)

5-Propargylamino-2′-deoxyuridine (1) (1.3 g, 4.6 mmol) and N-trifluoroacetyl-β-alanine succinimidyl ester (1.29 g, 4.6 mmol) were dissolved in DMF (100 mL) at ambient temperature. Triethylamine (0.46 g, 0.6 ml 4.6 mmol) was added and the solution stirred overnight at ambient temperature. The solvent was then removed under vacuum. The residue was then re-dissolved in dichloromethane:methanol (1:1) and eluted through a flash silica gel column (dichloromethane: methanol, 9:1). Removal of solvent from the appropriate fractions (R_{f}0.1, dichloromethane:methanol 9:1) afforded the title compound as a white foam (0.7 g, 34%). ¹HNMR (300 MHz, d₆-DMSO) δ 11.62 (1H, s, N³—H), 9.48 (1H, t, br, CF₃CONH), 8.48(1H, t, J5.4 Hz, propargyl NH), 8.15 (1H, s, H-6), 6.09 (1H, app t, J6.6 Hz, H-1′), 5.25 (1H, d, J4.5, 3′-OH), 5.10 (1H, t, J4.5 Hz, 5′-OH), 4.22 (1H, m, H-4′), 4.07 (2H, d, J5.1 Hz, propargyl CH2), 3.77 (1H, m, H-3′), 3.61-3.51 (2H, m, H-5′), 3.40-3.33(2H, β-ala CH₂CONH part obs. by HDO), 2.37 (2H, t, J6.9 Hz, gala CH₂NHTFA), 2.10 (2H, dd, J4.8 Hz, H-2′); ¹³C NMR (75.45 MHz, d₆-DMSO) δ 169.37, 161.61, 156.19 (q, J²C-F 35.9 Hz), 115.85 (q, J¹ C-F 286.5 Hz), 98.06, 89.45, 87.62, 84.70, 74.44, 70.23, 70.13, 61.02, 35.78, 33.80,28.58, 25.20, 8.75; MS (ES+) m/z 449 (M+H)⁺, 466(M+H2O)⁺.

### ii) 5-N-(β-alanyl)propargylamino-2′-deoxyuridine (3)

5-N-(N-Trifluoroacetyl-β-alanyl)propargylamino-2′-deoxyuridine (2) was dissolved in concentrated aqueous ammonia at ambient temperature. The solution was allowed to stir overnight at ambient temperature. The solvent was then removed under vacuum to give the title compound as a pale yellow foam (0.62 g, 100%). ¹H NMR (300 MHz, d₆-DMSO) δ 8.61 (1H, t, J5.1 Hz, propargyl NH), 8.16 (1H, s, H-6), 6.09 (1H, app t, J6.6H, H-1′), 4.21 (1H, m, H-4′), 4.10 (2H, d, J5.1 Hz, propargyl CH₂), 3.79 (1H, m, H-3′), 3.59 (2H, m, H-5′), 2.96 (2H, t, J6.9 Hz, β-ala CH₂), 2.44 (2H, t partly obs, β-ala CH₂), 2.10 (2H, m, H-2′); ¹³C NMR (75.45 MHz, d₆-DMSO) δ 169.23, 161.61, 149.41, 143.76, 97.98, 89.25, 87.64, 84.74, 74.60, 55.46,35.31, 32.36, 28.62, 25.20; MS (ES+) m/z 353(M+H)⁺.

### iii) 5-N-(N-Fluorenylmethyloxycarbonyl-Gly-Gly-Leu-β-alanyl)propargylamino-2′-deoxyuridine (4)

5-N-(β-alanyl)propargylamino-2′-deoxyuridine (3) 0.1 g (0.28 mmol) and N-fluorenylmethyloxycarbonyl-Gly-Gly-Leu succinimidyl ester (0.17 g, 0.3 mmol) were weighed into a round bottom flask and then dissolved in anhydrous DMF (1 mL). Triethylamine (0.061 g, 0.08 mL, 0.6 mmol) was then added and the reaction mixture stirred at ambient temperature. After 2.5 hours the solvents were removed under vacuum and the residue redissolved in dichloromethane-methanol (9:1) and eluted with dichloromethane-methanol (9:1 then 8:2). Collected and removed solvent from fractions containing R_{f}0.5 material (8:2 dichloromethane:methanol). Obtained the title compound as a white solid 0.04 g (18%). ¹H NMR (300 MHz, d₆-DMSO) δ 11.60 (1H, s, br, N³—H), 8.38 (1H, t, amide NH), 8.15 (1H, s, H-6), 7.95 (1H, t, amide NH), 7.90 (1H, d, amide NH), 7.88 (2H, d, J7.5 Hz, Fmoc), 7.70 (2H, d, J7.5 Hz, Fmoc), 7.62 (1H, t amide NH), 7.41 (2H, t, J7.5 Hz, Fmoc), 7.30 (2H, t, J7.5 Hz, Fmoc), 6.09 (1H, app t, J6.6 Hz, H-1′), 5.24 (1H, d, br, 3′-OH), 5.09 (1H, t, 5′-OH), 4.29-4.18 (4H, m), 4.05 (2H, m), 3.79-3.54 (5H, m), 2.25 (2H, m, β-ala CH2), 2.10 (2H, m, H-2′), 1.51 (1H, sept, leucyl CH₂CH(CH₃)₂), 1.42 (2H, m, leucyl CH₂CH(CH₃)₂), 0.84-0.78 (6H, 2d, leucyl CH₂CH(CH₃)₂); MS (ES+) m/z 802(M+H)⁺.

### iv) 5-N-(-Gly-Gly-Leu-β-alanyl)propargylamino-2′-deoxyuridine (5)

5-N-(-N-Fluorenylmethyloxycarbonyl-Gly-Gly-Leu-β-alanyl)propargylamino-2′-deoxyuridine (4) was dissolved in anhydrous DMF at ambient temperature. Piperidine was then added and the solution stirred at ambient temperature overnight. Solvents and volatile reagents were removed under vacuum. The product was used without further purification. MS (ES+) m/z 580(M+H)⁺.

### v) 5-N-[N-(6-Fluorescein-5(and 6)carboxamidohexanoyl)-Gly-Gly-Leu-β-alanyl]-propargylamino-2′-deoxyuridine (6)

5-N-(-Gly-Gly-Leu-β-alanyl)propargylamino-2′-deoxyuridine (5) (11.2 μmol) and 6-(fluorescein-5(and 6)carboxamidohexanoic acid succinimidyl ester (0.098 g, 16.8 μmol) were dissolved in DMF (0.2 mL) at ambient temperature. Triethylamine (3 μL, 22.4 μmol) was added and the solution allowed to stand at ambient temperature overnight. Solvents and volatile reagents were removed under vacuum. The residue was washed with dichloromethne-methanol-acetic acid 90:9:1 to remove unreacted dye and residual triethylamine. The solid residue was then dissolved in methanol-water and purified by reverse phase HPLC (isocratic water-methanol 1:1/C18 stationary phase). Obtained the title compound (5.3 μmol) as an orange powder after lyophilisation of the appropriate fractions. ¹H NMR (300 MHz, d₄-MeOH) δ8.47 (1H, s), 8.28 (1H, s), 8.20-8.00 (3H, m), 7.31 (1H, d), 7.10-7.00 (4H, m), 6.21 (1H, app t, H-1′), 4.6 (1H, m), 430 (2H, m), 4.21 (2H, d, propargyl CH₂), 3.9-3.6 (7H, m, glycyl α-H, H-5′, leucyl α-H), 2.5-2.1 (6H, m, H-2′, β-ala CH₂, caproamide CH₂CONH), 2.75-1.4 (12H, caproamide CH₂, leucyl CH₂CH(CH₃)₂), 0.80 (6H, 2d, leucyl CH₂CH(CH₃)₂); MS (ES+) m/z 1051(M+H)⁺; UV λₘₐₓ 498 nm (MeOH—NH₄OH, pH9).

### vi) 5-N-[N-(6-Fluorescein-5(and-6)carboxamidohexanoyl)-Gly-Gly-Leu-βalanyl]-propargylamino-2′-deoxyuridine Triphosphate

5-N-(N-Fluorenylmethyloxycarbonyl-Gly-Gly-Leu-β-alanyl)propargylamino-2′-deoxyuridine (4) may be converted to a dye-labelled triphosphate by using established triphosphate syntheses (for example, see K. Burgess & D. Cook, Chem. Rev. 2000, 100, 2047-2059 and references cited therein). The triphosphate of compound (4) could then be treated with piperidine under the same conditions as used to prepare compound (5) and then labelled as described above for the preparation of compound (6).

### EXAMPLE 2

### Protease Mediated Cleavage of 5-N-[N-(6-Fluorescein-5(and-6)carboxamidohexanoyl)-Gly-Gly-Leu-β-alanyl]-propargylamino-2′-deoxyuridine (FamHex-GGL-β-A2′dU)

FIG. 2 shows the hydrolytic cleavage of FamHex-GGL-β-A2′dU (6) by the proteolytic enzyme, subtilisin. Compound 6 is readily digested by subtilisin (Subtilopeptidase A, type VIII, Sigma Chemical Company, UK), which cleaves at the leucine residue, following 2 hours incubation at 37° C. at pH 7.5 to yield the nucleoside and dye-labelled products shown.

### EXAMPLE 3

### Synthesis of a Nucleotide with a Penicillin Amidase Cleavable Linker

FIG. 3 shows a reaction scheme for the preparation of a nucleotide with a penicillin amidase cleavable linker.

5-Hydroxymethyl-5′,3′-di-O-toluyl-2′-deoxyuridine (7) (prepared using established procedures (T. Ueda, Chemistry of Nucleosides and Nucleotides, Vol. 1.Ed. L. B. Towensend) and N-[α-thioethyl-N′-trifluoroacetylaminopropyl benzamide]phenylacetamide (8) (Flitsch et al., Tetrahedron Letters 1998, 39, 3819-3822 and references cited therein; Flitsch e al. WO 97/20855) may be combined in the presence of N-iodosuccinimide to give compound (9). Compound (9) may be converted to the intermediate (10) by treatment with sodium methoxide in methanol, followed by ethyl trifluoroacetate in methanol. Conversion of the nucleoside (10) to a triphosphate (11) may be achieved by using established triphosphate synthesis conditions (For examples see K. Burgess, D. Cook. Chem. Rev. 2000, 100, 2047-2059 and references cited therein). Labelling of the triphosphate with a reporter group may then be achieved by exposure of compound (11) to proprietary labelling reagents, such as 6-[Fluorescein-5(and-6)-carboxamidohexanoic acid succinimidyl ester, in a suitably buffered aqueous solution to give (12).

### EXAMPLE 4

### DNA Polymerase Assays

Incorporation of Fluorescently Labelled Deoxynucleotide Triphosphates by DNA Polymerases

### (i) Materials

The following olignucleotides (Interactiva Biotechnologie, Germany) were used for assaying nucleotide incorporation:

Primer Sequence:—

SEQ. ID No 1:
5′-TAA CTC ATT AAC AGG ATC-3′

Template Oligonucleotides:—

SEQ. ID No 2:
ATTCGCGGTATTCTGGTATGAAGCTTTTAGATCCTGTTAATGAGTTAGTA
SEQ. ID No 3:
ATTCGCGGTATTCTGGTATGAAGCTTTAAGATCCTGTTAATGAGTTAGTA
SEQ. ID No 4:
ATTCGCGGTATTCTGGTATGAAGCTTAAAGATCCTGTTAATGAGTTAGTA
SEQ. ID No 5
ATTCGCGGTATTCTGGTATGAAAAAAAAAGATCCTGTTAATGAGTTAGTA

Fluorescently labelled nucleotides were obtained from the following sources:—
Molecular Probes Inc:
Alexa Fluor 546-14-dUTP 1 mM in TE
Alexa Fluor 568-5-dUTP 1 mM in TE
Alexa Fluor 594-5-dUTP 1 mM in TE
   Nen, UK:
Fluorescein-12-dUTP
Coumarin-5-dUTP
Tetramethylrhodamine-6-dUTP
Texas Red-5-dUTP
Lissamine-5-dUTP
Napthofluorescein-5-dUTP
Fluorescein Chlorotriazinyl-4-dUTP
Pyrene-8-dUTP
Diethylaminocoumarin-5-dUTP
   Amersham Biosciences, UK:
Cy3 dUTP
Cy5 dUTP

### (ii) Oligonucleotide Labelling

Oligonucleotide 1 was labelled with [gamma-33P]ATP (Amersham Biosciences) using T4 Polynucleotide Kinase (Amersham Biosciences) in accordance with the manufacturer's protocol.

### (iii) Primer Extension Reactions

Each 20 μl primer extension reaction, assembled in 0.5 ml thermal cycling microfuge tube, contained at a final concentration 1×Thermosequenase™ buffer (Amersham Biosciences), 0.05 mM 33P labelled oligonucleotide 1, 0.125 mM template oligonucleotide, 0.125 mM dye-labelled nucleotide and 0.165 units of enzyme. The reactions were denatured at 95° C. for 1 minute before incubation at 48° C. for 45 minutes.

The reactions were terminated by the addition of 5 μl of stop buffer comprising 0.1% (w/v) Xylene Cyanol, 0.1% (w/v) Bromophenol blue and 80% Formamide. The reactions were heated to 90° C. for 3 minutes then chilled on ice.

A 14% denaturing polyacrylamide gel in 1×TBE (Sequagel, Flowgen Ltd, UK and Sigma Chemical Co., UK, respectively) was pre-run at 40 W constant power for 30 min in 1×TBE running buffer. An 8 μl aliquot of denatured reaction was then applied to each lane and the samples electrophoresed for 1.5 hours at 40 W constant power. The gel was exposed to a phosphor screen (Amersham Biosciences) for 1 hour and the image detected on the Storm™ Imager (Amersham Biosciences) in accordance with the manufacturers guidelines.

### (iv) Results

All the fluor-labelled nucleotides were incorporated by Thermosequenase. Templates 2, 3, 4 and 5 had 1, 2, 3, or 8 consecutive A residues respectively. The number of consecutive fluor-labelled nucleotides added was dependent upon the template and the fluor species and is summarised below:

| | |
|---|---|
| Fluor | predominant product with templates 3, 4 and 5 |
| Cy3 | One addition, with minor two additions |
| Cy5 | One addition, with minor two additions |
| Tetramethylrhodamine | One addition, with minor two additions |
| Coumarin | One addition, with a comparable amount of |
| | template with two additions |
| Alexa 546-14-dUTP | One addition, with a comparable amount of |
| | template with two additions |
| Fluorescein | One addition, with minor two additions |
| Texas Red | One addition, with very slight two additions |
| Lissamine | One addition, with very slight two additions |
| Alexa 568-5-dUTP | One addition |
| Alexa 594-5-dUTP | One addition |

Even when the template contained three or more consecutive dA residues (oligonucleotide 4 and 5), the number of nucleotides added did not noticeably exceed two bases. The efficiency of the second base addition appeared to be dependent on the label attached. Some fluors such as Coumarin appeared to demonstrate greater efficiency in adding two consecutive bases.

### EXAMPLE 5

### Protease Mediated Cleavage of 5-N-[N-(6-Fluorescein-5(and-6) carboxamido hexanoyl)-Gly-Gly-Leu-β-alanyl]-propargylamino-2′-deoxyuridine (6) (FamHex-GGL-β-A-2′dU)

A 10 μg aliquot of the substrate FAMHEX-GGL-β-A 2′dU (compound 6 above) was dissolved in 0.1M sodium acetate buffer pH 7.5 containing 5 mM calcium acetate. The substrate solution was digested at 37° C. for 2 hours in 200 μl in the presence of 0.5 units of subtilisin (Subtilopeptidase A, type VIII, Sigma Chemical Co. UK).

The resulting digest was ultrafiltered using a Microcon YM10 Concentrator (Milipore Ltd., UK) and the filtrate analysed by HPLC (Gilson 170, Gilson, UK) on a Sephasil™ peptide C-18 column (Amersham Biosicences, UK). Four major peaks of absorbance at 438 nm were observed in the substrate and product. These eluted at 29.8 (I), 32.6 (II), 26.7 (III) and 37.1 (IV) minutes on an acetonitrile gradient of 50% to 70%. The peaks were collected and analysed by MALDI-TOF mass spectrometry (Bruker Biflex II, Brucker, Germany). A matrix of 10 mg/ml 3-hydroxycinnamic acid in acetonitrile and 1% TFA was used for the analysis. The mass spectrometer was calibrated with 100 mM Bradykinin 1-7 (Sigma Chemical Co., UK).

The undigested substrate contained a predominant peak (I) that was shown by mass spectrometry to contain the intact substrate molecule. The remaining peaks corresponded to components of the intact molecule probably carried over from the synthesis. After substrate digestion with subtilisin, peak I was seen to diminish substantially and peak II demonstrated a corresponding increase in absorbance at 438 nm. The latter peak was shown to contain a molecular ion of 717 that corresponded to a fluor-linker moiety resulting from the hydrolysis of the substrate at the peptide bond on the carboxyl side of the leucine residue. These observations were consistent with the cleavage of the linker by the subtilisin enzyme. There was no observable change in peaks III and IV, suggesting that they do not act as substrates for the protease enzyme.

### EXAMPLE 6

### Preparation of Dye-Labelled Nucleosides with Protease Cleavable Linkers

Linker motifs suitable for use in protease cleavable linkers were prepared and identified using the following procedures.

### (i) General LibraryAmino Acid Coupling Procedure

A polystyrene resin loaded with 5-propargylamino-2′-deoxyuridine was prepared using standard solid phase chemical methods. The resin was distributed between 21 disposable filter vessels (10-20 mg per vessel). The filter tubes containing the resin were placed on a vacuum manifold, DCM was added to swell the resin and the excess was drained off. An microtag was added to the vessel for identification purposes. Solutions of Fmoc-AA-OH (or Fmoc-Ahx-OH), DIC and HOBt in DCM/DMF were prepared. The appropriate solution of activated amino acid was added, the reaction vessels, attached to the manifold, were placed horizontally on a flat bed shaker. The vessels were agitated for 3-3.5 hours. The vessels were drained of the reaction mixture and the resin was washed with: DMF (5×1 ml), DCM (5×1 ml), MeOH (5×1 ml), Et2O (3×1 ml), (volume of washings are shown per filter tube).

### (ii) General Library Fmoc Deprotection Procedure

DMF was added to the vessels to allow the resin to swell. After draining off the DMF 20% piperidine in DMF was added and the vessels left to agitate for 1 hour. The deprotection mixture was drained off and the resin was washed with: DMF (5×1 ml), DCM (5×1 ml), MeOH (5×1 ml), Et2O (3×1 ml).

Amino acid coupling and Fmoc deprotection procedures were repeated with different combinations of activated amino acids added to each tube to generate a library of compounds. Fmoc-Ahx-OH was the final amino acid to be added to all portions of the resin. After final Fmoc deprotection the compounds were labelled with dye and cleaved from the solid support according to the procedures described below.

### (iii) General Fluorphore Coupling Procedure

To the pre-swollen resin (DMF) was added a solution of fluorescein isothiocyanate isomer I and NEt3 in DMF (1 ml). The resin was shaken overnight and was then washed with DMF (5×1 ml), DCM (5×1 ml), MeOH (5×1 ml) and Et2O (5×1 ml).

### (iv) General Cleavage Procedure

5% TFA in DCM (0.2 ml) was added to resin (8 to 14 mg) and left to stand for 2 minutes. DCM (0.3 ml) was added and the resin was left to stand for 3 minutes before the DCM/TFA mixture was collected 5% TFA in DCM (0.2 ml) was added and the resin left to stand for 2 minutes. A drop of MeOH and DCM (0.3 ml) was added and the resin was left to stand for 3 minutes. This cleavage procedure was repeated (x˜3) to ensure maximum release of compound. An average loading of 0.430 mmole/g could be determined by HPLC.

### (v) Protease Cleavage Assay

All compounds prepared were assayed with a selection of the preferred proteases to determine the most suitable linker/protease combinations.

A solution of approximately 10 μg of nucleoside in water (5 μl) was mixed with 1 unit of protease (1-3 μl depending on enzyme stock solution) in the appropriate buffer for the enzyme. Final volumes of solutions were made up to 200 μl. Solutions were then held at enzyme optimum temperature for two hours and then centrifuged through 10,000 D molecular weight size exclusion membranes [Amicon Microcon YM-10] to remove the enzyme. The appropriate protease substrates reactions and standard control solutions were also prepared and treated in the same manner. All solutions were analysed using C18 reverse phase h.p.l.c (0.1%TFA/water:0.042%TFA/Acetonitrile, 95: 5-0:100 linear gradient). Successful cleavage of the linker was observed when several new products appeared on h.p.l.c., some of which possessed only the nucleoside chromophore.

### (vi) Preparation of Representative Dye-Labelled Nucleotide Triphosphate with a Protease Cleavable Linker

Preparation of a dye labelled nucleotide triphosphate incorporating a protease cleavable linker was performed as described below after selection of a suitable linker motif from the nucleoside library.

a) Preparation of Dye-Linker Group

The synthesis of an example of a dye-linker group, according to Formula II, is described below.

Cy5 carboxylic acid (10 mg, 0.17 mmol) [Amersham Biosciences] and N,N′N′-tetramethyl-O-(N-succinimidyl)uronium tetrafluoroborate (57 mg, 0.19 mmol) [Fluka] were weighed into an over-dried round-bottom flask. Anhydrous dimethylsulfoxide (250 μl) [Aldrich] was then added to dissolve the solids. Neat diisopropylethylamine (32 mg, 0.25 mmol, 43 μl) [Aldrich] was then added. The resulting solution was then stirred at ambient temperature for 4 hours. Thin layer chromatography (4:1 dichloromethane:methanol) showed the complete conversion of the starting material (Rf 0.2) to the N-hydroxysuccinimidyl ester (Rf 0.5). A solution of H2N-GlyGlyLeu-OH (42 mg, 0.17 mmol) [Bachem] was prepared by heating the peptide in anhydrous dimethylsulfoxide at 60° C. The solution was then allowed to cool to ambient temperature and was then added immediately to the solution of the Cy5-carboxylic acid N-hydroxysuccinimidyl ester. The reaction mixture was stirred at ambient temperature overnight. Analysis by thin layer chromatography (4:1 dichloromethane:methanol) showed the complete conversion of the active ester (Rf 0.5) to a new product which was immobile on thin layer chromatography. The majority of the solvents were then removed under vacuum to give the crude product as a dark blue oil. The oil was redissolved in methanol (80 μl). Half of the solution was then added to the top of a flash silica gel column and then eluted with 79:20:1 dichloromethane:methanol:acetic acid. Fractions were collected and those containing the predominant blue coloured component were pooled. The solvent was then removed under vacuum to give the pure Cy5-GlyGlyLeu conjugate as a dark blue solid. δ_{H} (CD₃OD, 300 MHz) 8.3(2H, 2t, vinylic), 7.8(2H, m, aromatic), 7.6-7.3(5H, m, aromatic), 6.6(1H, t, vinylic), 6.4(1H, d, vinylic), 6.2(1H, d, vinylic), 4.4(1H, m, leucine α-H), 4.1(2H, dd, —C₄H8CH2CONH), 4.0(1H, d, Gly a-H), 3.8(2H, s, Gly a-H), 3.7(1H, d, Gly a-H), 3.6(3H, s, CH3N+), 2.4(2H, dd, CH2N), 2.0-1.5(9H, m, 4xCH2, Leu CH(CH3)2), 0.85(6H, s, Leu CH(CH3)2); δ_{C} (CD3OD, 75.45 MHz) 177.5, 176.2, 174.3, 172.2, 156.6, 155.0, 145.9, 143.3, 143.1, 142.7, 142.1, 129.9, 128.0, 127.3, 126.9, 123.5, 121.2, 112.7, 110.8, 105.7, 104.1, 56.8, 55.8, 51.0, 45.2, 43.7, 42.9, 36.3, 31.4, 28.3, 27.8, 27.3, 26.1, 23.8, 22.2, 18.0, 13.1; λmax 642 nm.

b) Coupling of Dye-Linker Group to Nucleoside

Cy5-GlyGlyLeu-OH (3.1 mg, 3.9 μmol) and N,N,N′,N′-tetramethyl-O-(N-succinimidyl)uronium tetrafluoroborate (1.4 mg, 4.7 μmol)[Fluka] were weighed into a 1 mL plastic tube. Anhydrous dimethylsulfoxide (50 μL) [Aldrich] was then added, followed by diisopropylethylamine (0.74 mg, 5.8 μmol, 1 μl) [Aldrich]. The reaction mixture was agitated and then allowed to stand at ambient temperature for 1 hour. A solution of 5-allylamino-2′-deoxyuridine-5′-triphosphate triethylammonium salt (3.8 mg, 4.1 μmol) in anhydrous dimethylsulfoxide (50 μl) was added. The solution was agitated and then allowed to stand at ambient temperature. Monitoring of the reaction by reverse phase h.p.l.c showed the presence of a small amount of new material. The reaction mixture was diluted with 0.1M triethylammonium bicarbonate (TEAB) buffer and lyophilised to give a gummy residue. The residue was then re-dissolved in water (200 μl) and then eluted through a preparative reverse phase h.p.l.c column (0.1M TEAB-Acetonitdile, 95:5-0:100 linear gradient over 30 minutes). Fractions containing the new material were collected and lyophilised. Further purification was performed by ion-exchange h.p.l.c to give the desired product as a blue foam after lyophilisation. δ_{H} (D2O, 300 MHz) 7.9-7.1 (8H, m, aromatic), 6.4-5.9 (5H, m, vinylic), 6.1 (1H, obs, H-1′), 4.3-3.0(9H, m), 2.2(2H, m, H-2′), 2.0-1.5 (3H, m), 0.8 (6H, m); δP (D2O, 300 Hz) 6.3, −11.1, −21.7; λₘₐₓ 644 nm.

### (vii) Cleavage of Linker with Subtilisin

A solution of approximately 10 μg of nucleoside in water (5 μL) was mixed with 1 unit of subtilisin (2 μl of 0.5 U μl⁻¹ enzyme stock solution) in the appropriate buffer for the enzyme (193 μl). Solutions were then held at enzyme optimum temperature for two hours and then centrifuged through 10,000 D molecular weight size exclusion membranes [Amicon Microcon YM-10] to remove the enzyme. The appropriate subtilisin substrate reaction and standard control solutions were also prepared and treated in the same manner. The filtrates were analysed using C18 reverse phase h.p.l.c (0.1%TFA/water:0.042%TFA/Acetonitrile, 95:5-0:100 linear gradient). Successful cleavage of the linker was observed when new products at 4.2 and 25.2 minutes (starting material retention time=24.2 minutes). The peak at 4.2 minutes contained only the nucleotide chromophore.

1. A nucleoside comprising a base, a sugar and a reporter moiety which also functions to limit polymerase activity, wherein the reporter moiety is attached to the base of the nucleoside through a linkage group cleavable by a hydrolase enzyme, wherein said hydrolase enzyme is selected from the group consisting of esterases, phosphatases, peptidases, penicillin amidases, glycosidases and phosphorylases.

2. A compound of Formula I
R-L1-L2-L3-L4-L5-BASE-SUGAR (I)
wherein
R is a reporter moiety
L1 and L5 are optional linkage groups each containing one or more atoms comprising hydrocarbon chains which may also contain other atoms such as n, O and s;
L2 and L4 are optional linkage groups comprising 1 or more amino acid residues; and
L3 is a linkage group that is susceptible to enzymic hydrolysis by a hydrolase enzyme, wherein hydrolytic cleavage may be within the group or adjacent to the group and wherein said hydrolase enzyme is selected from the group consisting of esterases, phosphatases, peptidases, penicillin amidases, glycosidases and phosphorylases.

3. The compound of claim 2, wherein enzymic hydrolysis of the linkage group L3 produces an unstable moiety which undergoes chemical hydrolysis.

4. The compound of claim 2, wherein the base comprises purines or pyrimidines or analogues thereof.

5. The compound of claim 2, wherein the sugar comprises ribose or deoxy-ribose or analogues thereof.

6. The compound of claim 2, wherein a mono-, di- or triphosphate group is attached to the sugar.

7. The compound of claim 6, wherein a triphosphate group is attached to the sugar.

8. The compound of claim 2, wherein the hydrolase is a peptidase selected from the group consisting of subtilisin, proteinase K, elastase, neprilysin, thermolysin, papain, plasmin, trypsin, enterokinase and urokinase.

9. The compound of claim 2, wherein L3 is a peptide selected from the group consisting of alanine-alanine-alanine, alanine-alanine-leucine, glycine-leucine-serine, glycine-serine-alanine-alanine-leucine and glycine-alanine-glycine-leucine.

10. The compound of claim 2, wherein R is a fluorophore, selected from the group consisting of fluoresceins, rhodamines, coumarins, BODIPY® dyes, phenoxazine dyes, cyanine dyes, acridone dyes and squarate dyes.

11. A chemical intermediate of Formula II
R-L1-L2-L3-L4-L5 (II)
wherein
R is a reporter moiety
L1 and L5 are optional linkage groups each containing one or more atoms comprising hydrocarbon chains which may also contain other atoms such as N, O and S;
L2 and L4 are optional linkage groups comprising 1 or more amino acid residues, and
L3 is a linkage group that is susceptible to enzymic hydrolysis by a peptidase enzyme.

12. The chemical intermediate of claim 11, wherein L3 is selected from the group consisting of alanine-alanine-alanine, alanine-alanine-leucine, glycine-leucine-serine, glycine-serine-alanine-alanine-leucine and glycine-alanine-glycine-leucine.

13. The chemical intermediate of claim 11, wherein R is selected from the group consisting of fluoresceins, rhodamines, coumarins, BODIPY® dyes, phenoxazine dyes, cyanine dyes, acridone dyes and squarate dyes.

14. The compound 5-N-[N-(6-Fluorescein-5(and-6) carboxamidohexanoyl)-Gly-Gly-Leu-β-alanyl]-propargylamino-2′-deoxyuridine triphosphate.

15. A set of nucleotides wherein the set contains at least one compound of claim 6.

16. The set of nucleotides of claim 15, which includes the bases A, G, C and T and analogues thereof.

17-18. (canceled)

19. A method for nucleic acid molecule sequencing comprising the steps of:
a) immobilising a complex of a primer and a template to a solid phase
b) incubating with a polymerase in the presence of the compound of claim 6.

20. (canceled)

21. The method of claim 20, wherein the hydrolase enzyme is selected from the group consisting of esterases, phosphatases, peptidases, penicillin amidases, glycosidases and phosphorylases.

22. The method of claim 21, wherein the peptidase is selected from the group consisting of subtilisin, proteinase K, elastase, neprilysin, thermolysin, papain, plasmin, trypsin, enterokinase and urokinase.

23. (canceled)

24. The method of claim 19, wherein the incorporation of the compound is determined by the detection of a single reporter group attached to the compound.


SEQUENCE LISTING (5 sequences)

SEQ ID NO: 1 - DNA, 18 bp, ARTIFICIAL SEQUENCE
  SYNTHETIC OLIGONUCLEOTIDE
taactcattaacaggatc

SEQ ID NO: 2 - DNA, 50 bp, ARTIFICIAL SEQUENCE
  SYNTHETIC OLIGONUCLEOTIDE
attcgcggtattctggtatgaagcttttagatcctgttaatgagttagta

SEQ ID NO: 3 - DNA, 50 bp, ARTIFICIAL SEQUENCE
  SYNTHETIC OLIGONUCLEOTIDE
attcgcggtattctggtatgaagctttaagatcctgttaatgagttagta

SEQ ID NO: 4 - DNA, 50 bp, ARTIFICIAL SEQUENCE
  SYNTHETIC OLIGONUCLEOTIDE
attcgcggtattctggtatgaagcttaaagatcctgttaatgagttagta

SEQ ID NO: 5 - DNA, 50 bp, ARTIFICIAL SEQUENCE
  SYNTHETIC OLIGONUCLEOTIDE
attcgcggtattctggtatgaaaaaaaaagatcctgttaatgagttagta